(19) United States
(12) Patent Application Publication
(10) Pub. No.: US 2022/0211684 A1
(43) Pub. Date: Jul. 7, 2022

(54) ANTIFUNGAL AGENTS FOR CANDIDA AURIS DECOLONIZATION

(71) Applicant: Scynexis, Inc., Jersey City, NJ (US)

(72) Inventors: David A. Angulo Gonzalez, Palmetto Bay, FL (US); Stephen Andrew Barat, Mendham, NJ (US)

(21) Appl. No.: 17/610,440

(22) PCT Filed: May 12, 2020
(86) PCT No.: PCT/US2020/032547

§ 371 Date: Nov. 10, 2021

Related U.S. Application Data
(60) Provisional application No. 62/849,019, filed on May 16, 2019.

(51) Int. Cl. A61K 31/4439; A61K 31/4196; A61P 31/10

(52) U.S. Cl. CPC A61K 31/4439; A61P 31/10; A61K 31/4196

(57) ABSTRACT

Enfumafungin derivative triterpenoid antifungal compounds are used to decolonize fungi from anatomic areas of subjects colonized by fungi. The enfumafungin derivative triterpenoids (or pharmaceutically acceptable salts or hydrates thereof) are inhibitors of (1,3)-β-glucan synthesis and can decolonize Candida auris from body sites such as skin and mucosa. Subjects who would benefit from such decolonization include individuals colonized by Candida auris who previously suffered a Candida auris infection and favor relapse and/or may transmit the fungus to other individuals who may be susceptible.

### FIELD OF THE INVENTION

The present invention relates to the use of enfumafungin derivative triterpenoid antifungal compounds to decolonize subjects colonized by susceptible fungi. More particularly, the invention relates to the use of enfumafungin derivative triterpenoids (or pharmaceutically acceptable salts or hydrates thereof) that are inhibitors of (1,3)-β-D-glucan synthesis, to decolonize body sites from Candida auris in subjects in which such strategy may be beneficial. Candida auris is a fungus that can, for example, remain in the skin of those who previously have suffered an infection thereby. The uses of enfumafungin derivative triterpenoid antifungal compounds of the present invention as described herein include but are not limited to: decolonization of skin or mucosa in patients colonized by Candida auris who previously suffered a Candida auris infection and favor relapse and/or may transmit the fungus to other individuals who may be susceptible. Although it does not relate to decolonization of inanimate objects (e.g., floors, furniture, instruments) per se, the present invention can reduce the colonization of such objects by decolonizing subjects who would otherwise spread Candida auris by contact, which reduction has benefits particularly in settings such as hospitals, hospices, and nursing homes.

### BACKGROUND OF THE INVENTION

Fungal infections are a major healthcare problem and are most commonly manifested as invasive or systemic fungal disease (e.g., candidemia, invasive aspergillosis), localized fungal infections (e.g., pleural empyema and abscess localized in abdomen, brain, lung, etc.), and mucocutaneous infections (e.g., oral, esophageal, and vulvovaginal candidiasis). The type and scope of the infection depends on the virulence factors of the fungal pathogen, the host's defenses, and the anatomic areas involved.

Severe systemic or invasive fungal infections are more common in immune-compromised patients, such as patients receiving chemotherapy to treat malignancies, or receiving immunomodulatory agents to treat chronic inflammatory conditions, or suffering from immune deficiencies, either acquired or due to genetic disorders. Despite currently available antifungal therapies, systemic fungal infections are associated with a mortality rate of up to 50% or more, depending on the pathogen and the underlying condition of the patient.

Localized and systemic fungal infections often originate via dissemination of fungi from a local area where they normally colonize to an area that is normally sterile (e.g., abscess in abdominal cavity after bowel perforation or surgery) or from fungi entering the blood or lymphatic system that reaches a particular organ (e.g., lung, liver, spleen) and develops into a fungemia or deep-seated infection. Fungi colonization for the purpose of this application means the presence of fungi in an anatomic area in which there is not a clinically identifiable host inflammatory reaction caused by the presence of the fungi (that is, the fungi is not causing an infection or the symptoms of an infection). Fungi colonization in susceptible individuals may facilitate the establishment of an infection by the colonizer pathogen and may facilitate spreading of the pathogen to other individuals. This can become particularly problematic when dealing with fungi that are not easily treated, have developed resistance to antifungal agents, and/or that lead to high rates of mortality.

Candida auris is a multidrug-resistant health care-associated fungal pathogen that has emerged as a challenge globally. Recent reports highlight ongoing challenges due to organism misidentification, high rates of antifungal drug resistance, and significant patient mortality. The predilection of Candida auris for transmission within and between health care facilities, possibly promoted by virulence factors that facilitate skin colonization and environmental persistence, is unique among Candida species.

Candida auris frequently causes prolonged colonization of patients' skin and contamination of surrounding environments, resulting in nosocomial outbreaks in hospitals and long-term care facilities. Clinicians, infection prevention and control practitioners, and public health officials are currently facing how to mitigate the threat posed by this pathogen. Generally, patients with symptomatic disease should be treated with an antifungal agent immediately, but the optimal management of patients colonized by Candida auris is still not well defined. However, it is recognized that patients colonized by Candida auris may be at increased risk of developing a symptomatic infection and that they may play an important role in the transmission of the pathogen to other susceptible individuals.

The Center for Disease Control (CDC) considers Candida auris to present a serious global health threat. The CDC is concerned about Candida auris for a number of reasons. The pathogen is often multidrug-resistant, meaning that it is resistant to multiple antifungal drugs commonly used to treat Candida infections. Further, it is difficult to identify with standard laboratory methods, and it can be misidentified in labs without specific technology—and misidentification may lead to inappropriate management. Moreover, the pathogen has caused outbreaks in healthcare settings, and it is important to quickly identify Candida auris in hospitalized patients so that healthcare facilities can take special precautions to stop its spread. On its webpage https://www.cdc.gov/fungal/candida-auris/fact-sheets/c-auris-colonization.html, the CDC addresses particular challenges posed by Candida auris colonization, mentioning that the pathogen can spread from one patient to another in hospitals and nursing homes. Patients can carry Candida auris somewhere on their body with the fungus not making them sick. When individuals in hospitals and nursing homes are colonized, Candida auris can easily spread from them to other people nearby, or to nearby objects and then to other people. While a simple test can be conducted to see whether a person is colonized with the fungus, individuals having Candida auris somewhere on their body may not have an infection or symptoms of infection, and may not even be aware that they pose a risk to and can spread Candida auris to others. In addition, persons colonized with Candida auris might later get sick from the fungus themselves, so healthcare providers must consider taking extra steps to prevent infection. The CDC recommends placing patients colonized by Candida auris in isolation with contact precautions, increasing the cost of management of these cases for the healthcare system.

The investigation of the first seven cases of Candida auris infection identified in the United States, which occurred between May 2013 and August 2016, showed colonization with Candida auris on skin and other body sites weeks to months after their initial infection, which could lead to contamination of the healthcare environment and pose a risk of continuous transmission. (Vallabhaneni S, Kallen A, Tsay S, et al., Investigation of the first seven reported cases of Candida auris, a globally emerging invasive, multidrug-resistant fungus—United States, May 2013-August 2016. Morb Mortal Wkly Rep 2016; 65:1234-1237. DOI: http://dx.doi.org/10.15585/mmwr.mm6544e1.) Currently recommended treatment options for Candida auris infection, such as echinocandins, have not prevented patients from remaining colonized by the fungus, particularly in the skin. And considering that Candida auris often is resistant to other antifungal agents such as azoles and polyenes, these other agents do not provide a suitable alternative for decolonization, either. Azoles, for example, do not decolonize Candida auris from anatomic areas colonized by the fungus, despite their ability to achieve high concentrations in the tissues. At present, the CDC does not recommend antifungal treatment of Candida auris identified from noninvasive sites (such as respiratory tract, urine, and skin colonization) when there is no evidence of infection. (https://www.cdc.gov/fungal/candida-auris/c-auris-treatment.html.)

Decolonization strategies with topical 2% aqueous chlorhexidine wipes have been attempted in a hospital reporting outbreaks. (Ruiz-Gaitan A et al., An outbreak due to Candida auris with prolonged colonization and candidaemia in a tertiary care European hospital, Mycoses 61:498-505 (2018). https://onlinelibrary.wiley.com/doi/epdf/10.1111/myc.12781.) A significant limitation of topical antiseptics is their inability to reach all skin and mucosal surfaces efficiently or at all. Moreover, the need to apply topical antiseptics up to several times a day can be bothersome.

Enfumafungin is a hemiacetal triterpene glycoside that is produced in fermentations of a Hormonema spp. associated with living leaves of Juniperus communis (U.S. Pat. No. 5,756,472; Pelaez et al., Systematic and Applied Microbiology, 23:333-343 (2000); Schwartz et al., JACS, 122: 4882-4886 (2000); Schwartz, R. E., Expert Opinion on Therapeutic Patents, 11(11): 1761-1772 (2001)). Enfumafungin is one of the several triterpene glycosides that have in vitro antifungal activities. The mode of the antifungal action of enfumafungin and other antifungal triterpenoid glycosides was determined to be the inhibition of fungal cell wall glucan synthesis by their specific action on (1,3)-β-D-glucan synthase (Onishi et al., Antimicrobial Agents and Chemotherapy, 44: 368-377 (2000); Pelaez et al., (2000)). 1,3-β-D-glucan synthase remains an attractive target for antifungal drug action because it is present in many pathogenic fungi and therefore affords a broad antifungal spectrum. In addition, because there is no mammalian counterpart to (1,3)-β-D-glucan synthase, the enfumafungin derivatives described herein have little or no mechanism-based toxicity. The triterpenoid compound derivatives of enfumafungin used according to this invention have demonstrated activity against fungal isolates of Candida spp., including those isolates that are resistant to azoles or other glucan synthase inhibitors (e.g., lipopeptide agents such echinocandins), indicating that the biological and molecular target of the enfumafungin derivatives is different from that of other glucan synthase inhibitors.

Various enfumafungin derivatives have been disclosed, e.g., in International Patent Publication Nos. WO 2007/126900 and WO 2007/127012. Certain representatives of these enfumafungin derivatives can be administered orally, have shown antifungal activity against Candida species, and have shown adequate distribution into tissues, including skin.

Ibrexafungerp (also referred as SCY-078) has shown in vitro activity against Candia auris. The in vitro susceptibility of SCY-078 against a collection of 100 isolates of Candida auris was reported by Berkow et al. Included in the study were isolates from each of the four known clades of Candida auris, originating from countries all over the world, including India, Pakistan, Colombia, South Africa, and the United States. All isolates were subjected to broth microdilution according to the standards of the Clinical and Laboratory Standards Institute reference methodology M27-A3. The distribution of MIC values of SCY-078 ranged from 0.0625 micrograms/ml to 2 micrograms/ml. The overall mode was 1 micrograms/ml, and the MIC₅₀ and MIC₉₀ were 0.5 micrograms/ml and 1 micrograms/ml, respectively. (Berkow E L, Angulo D, Lockhart S R, In vitro activity of a novel glucan synthase inhibitor, SCY-078, against clinical isolates of Candida auris, Antimicrob Agents Chemother 61:e00435-17 (2017) https://doi.org/10.1128/AAC.00435-17.)

Persistent skin colonization by Candida auris has been reported after treatment with currently available systemic antifungal agents, and this phenomenon is associated with increased risk of developing an infection due to Candida auris and increased risk of transmission of the pathogen which may facilitate outbreaks. There is a need in the art to be able to decolonize Candida auris from anatomic areas of a subject, especially skin or mucosa, to reduce the risk of relapse of the disease in the subject as well as to help prevent dissemination of the fungus and potential outbreaks.

### SUMMARY OF THE INVENTION

The present invention addresses the need in the art for decolonization, especially of skin or mucosa, in patients colonized by Candida auris who previously suffered a Candida auris infection and favor relapse and/or may transmit the fungus to other individuals who may be susceptible. In such situations, potent and effective antifungal decolonization is particularly necessary.

The enfumafungin derivatives described herein are unexpectedly able to significantly reduce Candida auris burden in the skin after oral administration and would be a useful strategy to prevent transmission and limit the risk for Candida auris outbreaks. The enfumafungin derivatives described herein are glucan synthase inhibitors that exhibit a combination of attributes—including high concentrations in skin (preferably >10 fold greater than plasma concentrations as demonstrated in rat C¹⁴ studies), reduction of Candida auris skin fungal burden (preferably at least 1 log reduction), potent antifungal activity against Candida auris (including echinocandin-resistant strains), and oral bioavailability—that position them to be an optimal solution for the need in the art for an agent to decolonize Candida auris from anatomic areas of a subject. Unexpectedly, these attributes contrast with those of echinocandins, glucan synthase inhibitors with similar mechanism of action but that do not achieve concentrations in skin that are higher than plasma (Felton T et al., Tissue Penetration of Antifungal Agents, Clin. Microbiol. Rev. 2014, 27(1):68), are not orally bioavailable, and for which colonization with Candida auris has been detected at multiple body sites, including nares, groin, axilla, and rectum for 3 months or more after initial intravenous echinocandin treatment (Jeffery-Smith A et al., Candida auris: a Review of the Literature, Clin Microbiol Rev. 2017 Nov. 15; 31(1). pii: e00029-17. doi: 10.1128/CMR.00029-17. Print 2018 January Review).

Applications of this invention include but are not limited to the ability to decolonize subjects that have skin or mucosa Candida auris colonization. Colonized subjects who may benefit from the present invention include but are not limited to: those who have suffered a Candida auris systemic infection and survived but who remain colonized and are at risk of a relapse of the systemic infection (e.g., are immuno-compromised); subjects who have been colonized by being in contact with infected individuals and may be at risk of developing a systemic Candida auris infection; colonized subjects who are being managed under isolation with contact precautions in special hospital rooms (decolonization will reduce their need for such special and costly management and precautions); colonized subjects residing in places in which contact with other individuals would be common (e.g., nursing homes) and could spread the pathogen to other susceptible individuals; colonized health-care workers who could spread the pathogen to other susceptible individuals; and colonized subjects who preferably should be decolonized prior to a procedure such as surgery.

The present invention provides the use of a compound of Formula (I), or a pharmaceutically acceptable salt or hydrate thereof:

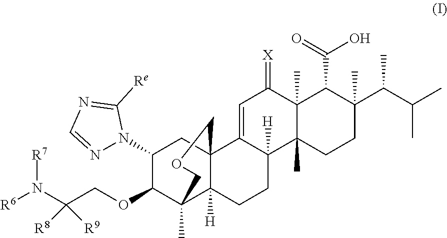

wherein:

X is O or H, H;

R^{e} is C(O)NR^{f}R^{g} or a 6-membered ring heteroaryl group containing 1 or 2 nitrogen atoms wherein the heteroaryl group is optionally mono-substituted on a ring carbon with fluoro or chloro or on a ring nitrogen with oxygen;

R^{f}, R^{g}, R⁶ and R⁷ are each independently hydrogen or C₁-C₃ alkyl;

R⁸ is C₁-C₄ alkyl, C₃-C₄ cycloalkyl or C₄-C₅ cycloalkyl-alkyl;

R⁹ is methyl or ethyl; and

R⁸ and R⁹ are optionally taken together to form a 6-membered saturated ring containing 1 oxygen atom,

to decolonize Candida auris from an anatomic area of a subject colonized by Candida auris. Anatomic areas of a subject that can be decolonized of Candida auris include but are not limited to skin and mucosa. Ibrexafungerp (SCY-078) is a preferred compound of Formula (I).

The invention also provides methods of decolonizing Candida auris from an anatomic area of a subject colonized by Candida auris, by administering to the subject the compound of Formula (I) or a pharmaceutically acceptable salt or hydrate thereof. In preferred methods, Candida auris is decolonized from the skin of a human subject. In preferred methods, ibrexafungerp as a compound of Formula (I) is administered orally to a human subject. Further, the invention provides the use of a compound of Formula (I) or a pharmaceutically acceptable salt or hydrate thereof in the preparation of a medicament for decolonizing Candida auris from an anatomic area of a subject colonized by Candida auris.

### DETAILED DESCRIPTION OF THE INVENTION

Decolonization strategies are particularly important: when the colonizing pathogen can cause life threatening infections, as is the case with Candida auris which has been associated with a mortality rate of ˜60%; and/or when the pathogen is resistant to antimicrobials, as is the case with Candida auris which is often reported to be resistant to antifungal agents currently available; and/or when the pathogen can be transmitted from person to person and cause outbreaks, which again is the case with Candida auris.

Topical application of antiseptics has been attempted as a decolonization strategy in subjects colonized by Candida auris, but this approach has limitations including: not reaching all body areas that may act as a Candida auris reservoir (e.g., mucosa, ear canal); and, due to such antiseptics having broad antimicrobial properties, affecting the skin's normal bacterial microbiome, increasing the risk of dysbiosis. Systemic antifungal agents such as echinocandins have been reported to be efficacious in treating systemic Candida auris disease (e.g., in the blood), but patients have been reported to remain colonized after treatment, particularly in the skin and mucosa, indicating that echinocandins may not be effective in achieving decolonization of colonized individuals. Additionally, echinocandins are only available intravenously and their use in decolonizing subjects that are not in a hospital setting (such as subjects in a home or nursing facilities) would be impractical.

An optimal decolonization agent should: have activity against the pathogen intended to be decolonized; cause minimal disruption to other colonizers that are part of the normal microbiome; achieve adequate concentrations in the intended tissues; remain active in those tissues for a period of time that will allow practical administration (e.g., BID, QD, once every other day, once every 3 days, etc.); and particularly for decolonization of the skin, should not have strong binding to keratin that may prevent the agent from being available for exhibiting its antifungal activity.

It has been found that the enfumafungin-derived triterpenoid ibrexafungerp (SCY-078)—a representative compound of enfumafungin derivatives described herein—surprisingly exhibits unique properties that would result in effective Candida auris decolonization in body sites (e.g., skin) of a human subject. Ibrexafungerp exhibits high concentrations in skin, reduction of Candida auris skin fungal burden, potent antifungal activity against Candida auris (including echinocandin-resistant strains), and oral bioavailability; these attributes contrast with those of echinocandins, glucan synthase inhibitors with similar mechanism of action but that do not achieve concentrations in skin that are higher than plasma, are not orally bioavailable, and for which colonization with Candida auris has been detected at multiple body sites, including nares, groin, axilla, and rectum for 3 months or more after initial intravenous echinocandin treatment. Ibrexafungerp, an antifungal agent, can be administered orally to decolonize Candida auris from, for example, the skin, to reduce the risk of the individual of having a relapse of the disease as well as to help prevent further dissemination of the fungus and potential outbreaks. This strategy, along with, for example, proper infection control in a hospital setting, can have a major impact on limiting the transmission of the disease, the cost of management, and ultimately the associated mortality.

Ibrexafungerp surprisingly exhibited significant activity to reduce Candida auris burden in animal models of Candida auris skin infection, supporting the use of this drug as a systemic antifungal agent effective in Candida auris skin decolonization in human subjects. In addition, ibrexafungerp showed good oral bioavailability (e.g., estimated in humans at >20%) and extensive tissue distribution following oral administration in mice and rats, achieving exposure in the skin that is 12 to 18 fold higher than the exposure in plasma (measured as Area Under the Curve) (Wring S, Borroto-Esoda K, Solon E, and Angulo D, SCY-078, a Novel Fungicidal Agent, Demonstrates Distribution to Tissues Associated with Fungal Infections during Mass Balance Studies with Intravenous and Oral [¹⁴C]SCY-078 in Albino and Pigmented Rats, Antimicrob Agents Chemother, 2019 Jan. 29; 63(2). pii: e02119-18. doi: 10.1128/AAC.02119-18. Print 2019 February PMID: 30478166). Such features are important for the treatment and prevention of fungal infections, as well as for achieving decolonization of susceptible fungal pathogens such as Candida auris.

Ibrexafungerp is a glucan synthase inhibitor, with a mechanism of action similar to that of the echinocandins but with a different chemical structure and a large volume of distribution; without intending to be bound by theory: it is believed that such properties allow ibrexafungerp to achieve appropriate concentrations in relevant tissues such as skin and mucosa, after oral administration, that inhibit the growth of Candida auris and result in preferably at least 1 fold the exposure observed in plasma and more preferably >2 fold or >5 fold or >10 fold the exposures observed in plasma. Additionally, other properties of the compound, which are not fully elucidated yet, may favor retention of the compound in an active form in tissues such as skin and mucosa, contributing to its antifungal effect in these tissues—making ibrexafungerp particularly relevant for decolonization strategies against a multi-drug resistant pathogen such us Candida auris. Ibrexafungerp does not have clinically relevant antibacterial properties and would not be expected to cause a deleterious effect on normal bacterial microbiome of the skin and mucosa.

The present invention provides the use of a compound of Formula (I), or a pharmaceutically acceptable salt or hydrate thereof:

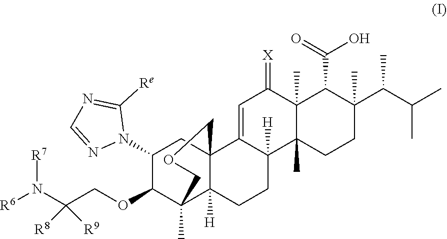

wherein:

X is O or H, H;

R^{e} is C(O)NR^{f}R^{g} or a 6-membered ring heteroaryl group containing 1 or 2 nitrogen atoms wherein the heteroaryl group is optionally mono-substituted on a ring carbon with fluoro or chloro or on a ring nitrogen with oxygen;

R^{f}, R^{g}, R⁶ and R⁷ are each independently hydrogen or C₁-C₃ alkyl;

R⁸ is C₁-C₄ alkyl, C₃-C₄ cycloalkyl or C₄-C₅ cycloalkyl-alkyl;

R⁹ is methyl or ethyl; and

R⁸ and R⁹ are optionally taken together to form a 6-membered saturated ring containing 1 oxygen atom,

to decolonize Candida auris from an anatomic area of a subject, such as a human subject, colonized by Candida auris. Anatomic areas of a subject that can be decolonized of Candida auris include but are not limited to skin and mucosa. Ibrexafungerp (SCY-078) is a preferred compound of Formula (I).

The present invention also provides the use of a compound of Formula (Ia), or a pharmaceutically acceptable salt or hydrate thereof:

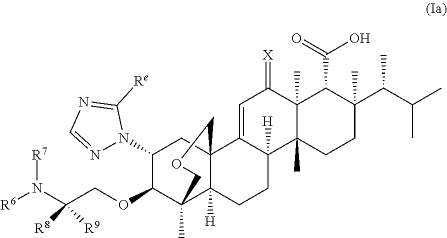

wherein the substituents are as provided for in Formula (I), to decolonize Candida auris from an anatomic area of a subject, such as human subject, colonized by Candida auris.

In embodiment 1: X is H, H, and the other substituents are as provided in Formula (I).

In embodiment 2: R^{e} is either pyridyl or pyrimidinyl optionally mono-substituted on a ring carbon with fluoro or chloro or on a ring nitrogen with oxygen, and the other substituents are as provided in embodiment 1 or in Formula (I).

In embodiment 3: R^{e} is 4-pyridyl and the other substituents are as provided in embodiment 1 or in Formula (I).

In embodiment 4: R^{e} is C(O)NH₂ or C(O)NH(C₁-C₃ alkyl) and the other substituents are as provided in embodiment 1 or in Formula (I).

In embodiment 5: R⁸ is C₁-C₄ alkyl and R⁹ is methyl; and the other substituents are as provided in embodiment 1, 2, 3, or 4, or in Formula (I).

In embodiment 6: R⁸ is t-butyl, R⁹ is methyl; and the other substituents are as provided in embodiment 1, 2, 3, or 4, or in Formula (I).

In embodiment 7: R⁶ and R⁷ are each independently hydrogen or methyl and the other substituents are as provided in embodiment 1, 2, 3, 4, 5, or 6, or in Formula (I).

In embodiment 1′: X is H, H, and the other substituents are as provided for in Formula (Ia).

In embodiment 2′: R^{e} is either pyridyl or pyrimidinyl optionally mono-substituted on a ring carbon with fluoro or chloro or on a ring nitrogen with oxygen, and the other substituents are as provided in embodiment 1′ or in Formula (Ia).

In embodiment 3′: R^{e} is 4-pyridyl and the other substituents are as provided in embodiment 1′ or in Formula (Ia).

In embodiment 4′: R^{e} is C(O)NH₂ or C(O)NH(C₁-C₃ alkyl) and the other substituents are as provided in embodiment 1′ or in Formula (Ia).

In embodiment 5′: R⁸ is C₁-C₄ alkyl and R⁹ is methyl; and the other substituents are as provided in embodiment 1′, 2′, 3′, or 4′, or in Formula (Ia).

In embodiment 6′: R⁸ is t-butyl, R⁹ is methyl; and the other substituents are as provided in embodiment 1′, 2′, 3′, or 4′, or in Formula (Ia).

In embodiment 7′: R⁶ and R⁷ are each independently hydrogen or methyl and the other substituents are as provided in embodiment 1′, 2′, 3′, 4′, 5′, or 6′, or in Formula (Ia).

In preferred embodiments, the present invention provides the use of a compound of Formula (II):

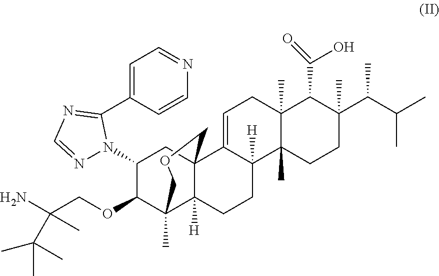

which is (1S,4aR,6aS,7R,8R,10aR,10bR,12aR,14R,15R)-15-[[2-amino-2,3,3-trimethylbutyl]oxy]-8-[(1R)-1,2-dimethylpropyl]-14-[5-(4-pyridinyl)-1H-1,2,4-triazol-1-yl]-1,6,6a,7,8,9,10,10a,10b,11,12,12a-dodecahydro-1,6a,8,10a-tetramethyl-4H-1,4a-propano-2H-phenanthro[1,2-c]pyran-7-carboxylic acid,

or a pharmaceutically acceptable salt or hydrate thereof, to decolonize Candida auris from an anatomic area of a subject, such as a human subject, colonized by Candida auris.

In other preferred embodiments, the present invention provides the use of a compound of Formula (IIa) (herein referred to as ibrexafungerp or SCY-078):

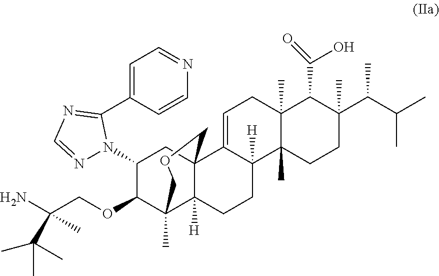

which is (1S,4aR,6aS,7R,8R,10aR,10bR,12aR,14R,15R)-15-[[(2R)-2-amino-2,3,3-trimethylbutyl]oxy]-8-[(1R)-1,2-dimethylpropyl]-14[5-(4-pyridinyl)-1H-1,2,4-triazol-1-yl]-1,6,6a,7,8,9,10,10a,10b,11,12,12a-dodecahydro-1,6a,8,10a-tetramethyl-4H-1,4a-propano-2H-phenanthro[1,2-c]pyran-7-carboxylic acid,

or a pharmaceutically acceptable salt or hydrate thereof, to decolonize Candida auris from an anatomic area of a subject, such as a human subject, colonized by Candida auris.

In preferred embodiments, the phosphate salt of a compound of Formula (I), (Ia), (II), or (IIa) is used or administered as described herein.

In preferred embodiments, the citrate salt of a compound of Formula (I), (Ia), (II), or (IIa) is used or administered as described herein.

The present invention also provides the use of a pharmaceutical composition comprising the compound of Formula (I), (Ia), (II), or (IIa), or a pharmaceutically acceptable salt or hydrate thereof, and a pharmaceutically acceptable carrier, adjuvant, or vehicle, to decolonize Candida auris from an anatomic area of a subject, such as a human subject, colonized by Candida auris.

The present invention further provides methods of decolonizing Candida auris from an anatomic area of a subject, such as a human subject, colonized by Candida auris, by administering to the subject the compound of Formula (I), (Ia), (II), or (IIa), or a pharmaceutically acceptable salt or hydrate thereof. The invention provides methods of decolonizing Candida auris from an anatomic area of a subject, such as a human subject, colonized by Candida auris, by administering a pharmaceutical composition comprising the compound of Formula (I), (Ia), (II), or (IIa), or a pharmaceutically acceptable salt or hydrate thereof, and a pharmaceutically acceptable carrier, adjuvant, or vehicle. In preferred methods, Candida auris is decolonized from the skin of a human subject. In preferred methods, ibrexafungerp is administered orally to a human subject. Further, the invention provides the use of a compound of Formula (I), (Ia), (II), or (IIa), or a pharmaceutically acceptable salt or hydrate thereof in the preparation of a medicament for decolonizing Candida auris from an anatomic area of a subject, such as a human subject, colonized by Candida auris.

In the description of compounds in the embodiments set forth above, indicated substitutions are included only to the extent that the substituents provide stable compounds consistent with the definition.

The compounds of Formula (I), (Ia), (II), and (IIa), and pharmaceutically acceptable salts and/or hydrate forms thereof, have antimicrobial (e.g., antifungal) activities against Candida auris and other fungi.

In view of their antifungal activity, compounds of Formula (I), (Ia), (II), and (IIa), and pharmaceutically acceptable salts and/or hydrate forms thereof, would be useful to decolonize Candida auris from anatomic parts or areas of a human subject colonized by Candida auris. Candida auris colonization is more commonly reported in the skin, and respiratory, gastrointestinal, and urinary tract mucosa. Colonized subjects who may benefit from the present invention include but are not limited to: those who have suffered a Candida auris systemic infection and survived but who remain colonized and are at risk of a relapse of the systemic infection (e.g., are immuno-compromised); subjects who have been colonized by being in contact with infected individuals and may be at risk of developing a systemic Candida auris infection; colonized subjects who are being managed under isolation with contact precautions in special hospital rooms (decolonization will reduce their need for such special and costly management and precautions); colonized subjects residing in places in which contact with other individuals would be common (e.g., nursing homes) and could spread the pathogen to other susceptible individuals; colonized health-care workers who could spread the pathogen to other susceptible individuals; and colonized subjects who preferably should be decolonized prior to a procedure such as surgery.

Through the uses and methods involving the compounds of Formula (I), (Ia), (II), and (IIa), and pharmaceutically acceptable salts and/or hydrate forms thereof, to decolonize Candida auris from human subjects, transmission of Candida auris can be reduced and prevented, and outbreaks of Candida auris can be better managed and prevented.

The compounds of Formula (I), (Ia), (II), and (IIa), and pharmaceutically acceptable salts and/or hydrate forms thereof, may be employed in the uses and methods described herein in combination with other environmental decontamination strategies to prevent transmission of Candida auris as well as to reduce the need by a colonized subject for isolation precautions.

The compounds of Formula (I), (Ia), (II), and (IIa), and pharmaceutically acceptable salts and/or hydrate forms thereof, may be employed in the uses and methods described herein to decolonize Candida auris from a human subject who previously suffered from a Candida auris infection and is at risk of relapse of such infections.

The uses and methods described herein can provide, at a body site colonized by Candida auris, a compound of Formula (I), (Ia), (II), or (IIa) (or pharmaceutically acceptable salts and/or hydrate forms thereof) at doses that are adequate to achieve a concentration effective to decolonize the site from Candida auris. Ibrexafungerp, in particular, would be highly effective for decolonization of Candida auris from skin in human subjects because clinical efficacy in Candida auris blood infections has been reported (Deven Juneja, Omender Singh, Bansidhar Tarai, and David Angulo Gonzalez, Successful Treatment of Two Patients with Candida auris Candidemia with the Investigational Agent, Oral Ibrexafungerp (formerly SCY-078) from the CARES Study, 13 Apr. 2019, ECCMID 2019, Amsterdam, The Netherlands, Abstract publication), and it can achieve exposures in skin that are higher than those achieved in plasma.

Through the uses and methods described herein, by which human subjects who would otherwise spread Candida auris by contact can be decolonized of Candida auris, the colonization of inanimate objects (e.g., floors, furniture, instruments) can indirectly be reduced, which reduction would have benefits particularly in settings such as hospitals, hospices, and nursing homes.

The compounds of Formula (I), (Ia), (II), and (IIa), and pharmaceutically acceptable salts and/or hydrate forms thereof, can be made according to the synthesis methods disclosed in U.S. Pat. No. 8,188,085, the contents of which are hereby incorporated by reference in their entirety.

As used herein, the term “alkyl” refers to any linear or branched chain alkyl group having a number of carbon atoms in the specified range. Thus, for example, “C₁₋₆ alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “cycloalkyl” refers to any cyclic ring of an alkane having a number of carbon atoms in the specified range. Thus, for example, “C₃₋₄ cycloalkyl” (or “C₃-C₄ cycloalkyl”) refers to cyclopropyl and cyclobutyl.

The term “cycloalkyl-alkyl” (or equivalently “alkyl-cycloalkyl”) as used herein refers to a system that includes an alkyl portion as described above and also includes a cycloalkyl portion as described above. Attachment to a “cycloalkyl-alkyl” (or “alkyl-cycloalkyl”) may be through either the cycloalkyl or the alkyl portion. The specified number of carbon atoms in “cycloalkyl-alkyl” systems refers to the total number of carbon atoms in both the alkyl and the cycloalkyl parts. Examples of C₄-C₅ cycloalkyl-alkyl include but are not limited to methylcyclopropyl, dimethylcyclopropyl, methylcyclobutyl, ethylcyclopropyl, cyclopropylmethyl, cyclopropylethyl and cyclobutylmethyl.

The term “halogen” (or “halo”) refers to fluorine, chlorine, bromine and iodine (alternatively referred to as fluoro, chloro, bromo, and iodo).

The term “or” as used herein denotes alternatives that may, where appropriate, be combined.

Unless expressly stated to the contrary, all ranges cited herein are inclusive. For example, a heterocyclic ring described as containing from “1 to 4 heteroatoms” means the ring can contain 1, 2, 3, or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, and so forth.

Any of the various cycloalkyl and heterocyclic/heteroaryl rings and ring systems defined herein may be attached to the rest of the compound at any ring atom (i.e., any carbon atom or any heteroatom) provided that a stable compound results. Suitable 5- or 6-membered heteroaromatic rings include, but are not limited to, pyridyl, pyrazinyl, pyrimidinyl, pyridazinyl and triazolyl.

A “stable” compound is a compound that can be prepared and isolated and whose structure and properties remain or can be caused to remain essentially unchanged for a period of time sufficient to allow use of the compound for the purposes described herein (e.g., therapeutic or prophylactic administration to a subject). Reference to a compound also includes stable complexes of the compound such as a stable hydrate.

As a result of the selection of substituents and substituent patterns, certain of the compounds of Formula (I), (Ia), (II), and (IIa) can have asymmetric centers and can occur as mixtures of stereoisomers, or as individual diastereomers, or enantiomers. Unless otherwise indicated, all isomeric forms of these compounds (and pharmaceutically acceptable salts and/or hydrate forms thereof), whether isolated or in mixtures, are within the scope of the present invention. Also included within the scope of the present invention are tautomeric forms of the compounds as depicted (and pharmaceutically acceptable salts and/or hydrate forms thereof).

When any variable occurs more than one time in any constituent or in Formula (I), (Ia), (II), or (IIa), its definition on each occurrence is independent of its definition at every other occurrence. Also, combinations of substituents and/or variables are permissible only if such combinations result in stable compounds.

The term “substituted” includes mono- and poly-substitution by a named substituent to the extent such single and multiple substitution (including multiple substitution at the same site) is chemically allowed. Unless expressly stated to the contrary, substitution by a named substituent is permitted on any atom in a ring (e.g., an aryl, a cycloalkyl, a heteroaryl, or a heterocyclyl) provided such ring substitution is chemically allowed and results in a stable compound.

A bond terminated by a wavy line is used herein to signify the point of attachment of a substituent group or partial structure. This usage is illustrated by the following example:

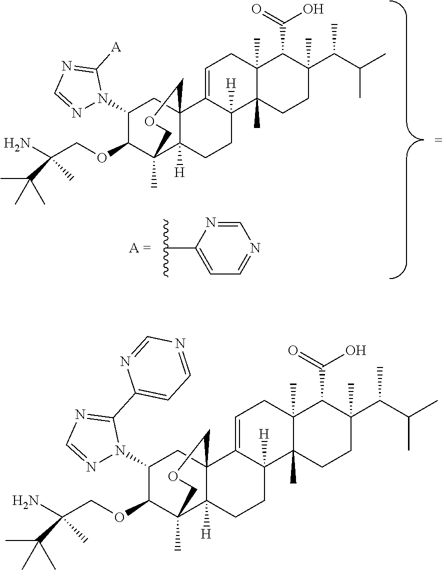

The compounds of Formula (I), (Ia), (II), and (IIa), and pharmaceutically acceptable salts and/or hydrate forms thereof, are also useful in the preparation and execution of screening assays for antifungal compounds. For example, the compounds are useful for isolating mutants, which are excellent screening tools for identifying further antifungal compounds.

The compounds of Formula (I), (Ia), (II), and (IIa) may be administered in the form of “pharmaceutically acceptable salts” or hydrates as appropriate. Other salts may, however, be useful in the preparation of the compounds or of their pharmaceutically acceptable salts. For example, when the compounds contain a basic amine group, they may be conveniently isolated as trifluoroacetic acid salts (e.g., following HPLC purification). Conversion of the trifluoroacetic acid salts to other salts, including pharmaceutically acceptable salts, may be accomplished by a number of standard methods known in the art. For example, an appropriate ion exchange resin may be employed to generate the desired salt. Alternatively, conversion of a trifluoroacetic acid salt to the parent free amine may be accomplished by standard methods known in the art (e.g., neutralization with an appropriate inorganic base such as NaHCO₃). Other desired amine salts may then be prepared in a conventional manner by reacting the free base with a suitable organic or inorganic acid. Representative pharmaceutically acceptable quaternary ammonium salts include the following: hydrochloride, sulfate, phosphate, carbonate, acetate, tartrate, citrate, malate, succinate, lactate, stearate, fumarate, hippurate, maleate, gluconate, ascorbate, adipate, gluceptate, glutamate, glucoronate, propionate, benzoate, mesylate, tosylate, oleate, lactobionate, laurylsulfate, besylate, caprylate, isetionate, gentisate, malonate, napsylate, edisylate, pamoate, xinafoate, napadisylate, hydrobromide, nitrate, oxalate, cinnamate, mandelate, undecylenate, and camsylate. Many of the compounds of Formula (I), (Ia), (II), and (IIa) carry an acidic carboxylic acid moiety, in which case suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The present invention includes within its scope the use of prodrugs of Formula (I), (Ia), (II), and (IIa). In general, such prodrugs will be functional derivatives of the compounds, which are readily convertible in vivo into the required compound. Thus, in the methods of treatment of the present invention, the term “administering” shall encompass the treatment of the various conditions described with the compound specifically disclosed or with a compound that converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of the compounds of Formula (I), (Ia), (II), and (IIa) include active species produced upon introduction of the compounds into the biological milieu.

The term “administration” and variants thereof (e.g., “administering” a compound) mean providing a compound (optionally in the form of a salt or hydrate thereof) or a prodrug of the compound to the subject in need of treatment. When a compound of Formula (I), (Ia), (II), and (IIa) or pharmaceutically acceptable salt thereof or a hydrate or prodrug thereof is provided in combination with a second active agent (e.g., other antifungal and/or antibacterial agents useful for treating fungal and/or bacterial infections), “administration” and its variants are each understood to include concurrent and sequential provision of the compound (or the salt, hydrate, or prodrug thereof) and of the other active agent.

As used herein, the term “composition” is intended to encompass a product comprising the specified ingredients, as well as any product that results, directly or indirectly, from combining the specified ingredients.

By “pharmaceutically acceptable” is meant that the ingredients of the pharmaceutical composition must be compatible with each other and not deleterious to the recipient thereof.

The term “subject” (alternatively referred to herein as “patient”) as used herein refers to an animal, preferably a mammal, most preferably a human, who has been the object of treatment, observation, or experiment.

The term “colonization” for the purpose of this application means the presence of a microorganism, such as a fungus, in an anatomic area in which there is not a clinically identifiable host inflammatory reaction caused by the presence of the microorganism (that is, the microorganism is not causing an infection or the symptoms of an infection). Colonization may be identified preferably by culture, but other methods used in the art are also acceptable to define colonization. Such other methods include but are not limited to: polymerase chain reaction (PCR) techniques, molecular sequencing, MALDI-TOF, microscopy or electron microscopy methods and magnetic resonance methods.

The term “decolonization” refers, in embodiments, to the reduction of a specific pathogen (e.g., Candida auris) burden in a specific body site (e.g., skin) in a sufficient magnitude that common culture techniques are no longer able to identify the pathogen. In other embodiments, “decolonization” refers to reduction of a specific pathogen burden in a sufficient magnitude that a desired benefit (e.g., limit of pathogen transmission, or reduction of the risk of infection relapse) is achieved.

The term “effective amount” as used herein means an amount of active ingredient or pharmaceutical agent that elicits the biological or medicinal response in a tissue, system, animal, or human that is being sought by a researcher, veterinarian, medical doctor, or other clinician. In one embodiment, the “effective amount” can be a therapeutically effective amount that alleviates the symptoms of the disease or condition being treated. In another embodiment, the “effective amount” can be a prophylactically effective amount for prophylaxis of the symptoms of the disease or condition being prevented or for reducing the likelihood of occurrence. The term can also refer to an inhibition effective amount of the enfumafungin derivative sufficient to inhibit (1,3)-β-D-glucan synthase and thereby elicit the response being sought.

References to “treat,” “treating,” “treatment,” and variants thereof, generally refer to a treatment that, after it is administered, results in resolution or improvement of one or more signs or symptoms associated with a fungal infection, or that results in eradication of the fungi responsible for an infection, or any combination of these outcomes.

For the purpose of decolonization, the compound of Formula (I), (Ia), (II), or (IIa) (optionally in the form of a salt or a hydrate) can be administered in conventional ways available for use in conjunction with pharmaceuticals.

For the purpose of decolonization, the compound of Formula (I), (Ia), (II), or (IIa) (optionally in the form of a salt or a hydrate) can be administered alone as an individual therapeutic agent or with one or more other antifungal agents (sequentially or concurrently) as a combination of therapeutic agents.

For the purpose of decolonization, the compound of Formula (I), (Ia), (II), or (IIa) (optionally in the form of a salt or a hydrate) can be administered with a pharmaceutical carrier selected on the basis of the chosen route of administration and standard pharmaceutical practice.

For example, the compounds of Formula (I), (Ia), (II), and (IIa), and pharmaceutically salts and/or hydrate forms thereof, can be administered by one or more of the following routes: orally, parenterally (including subcutaneous injections, intravenous, intramuscular, intra-lesion injection or infusion techniques), by inhalation (e.g., nasal or buccal inhalation spray, aerosols from metered dose inhalator, and dry powder inhalator), by nebulizer, ocularly, topically, transdermally, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose.

Further description of methods suitable for use in preparing pharmaceutical compositions and of ingredients suitable for use in said compositions is provided in Remington's Pharmaceutical Sciences, 20^{th} edition, edited by A. R. Gennaro, Mack Publishing Co., 2000.

The compounds of Formula (I), (Ia), (II), and (IIa), and pharmaceutically acceptable salts and/or hydrate forms thereof, can be administered, e.g., orally or intravenously, in a dosage range of, for example, 0.01 to 1000 mg/kg of mammal (e.g., human) body weight per day in a single dose or in divided doses. An example of a dosage range is 0.1 to 500 mg/kg body weight per day orally or intravenously in a single dose or in divided doses. Another example of a dosage range is 1 to 50 mg/kg body weight per day orally or intravenously in single or divided doses. For oral administration, the compositions can be provided in the form of tablets or capsules containing, for example, 1.0 to 1000 milligrams of the active ingredient, particularly 1, 5, 10, 15, 20, 25, 50, 75, 100, 150, 200, 250, 300, 400, 500, 600, 750, and 1000 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. For example, in embodiments, a pharmaceutically acceptable salt of the compound of Formula (IIa) is administered to a subject to provide a total daily dose of 150 to 750 mg of the compound of Formula (IIa). In certain embodiments, a total daily dose of 150 mg, a total daily dose of 300 mg, or a total daily dose of 500 mg, or a total daily dose of 600 mg, or a total daily dose of 750 mg of the compound of Formula (IIa) is administered; the total daily dose may be administered on a once-daily basis or it may be divided such as for BID (twice daily) dosing or TID (thrice daily) dosing or once every other day dosing or once every 3 days dosing.

The present invention provides methods for decolonization of Candida auris from one or more anatomic parts or areas of a human subject colonized by Candida auris, comprising administering an amount of a compound of Formula (I), (Ia), (II), or (IIa) (or a pharmaceutically acceptable salt or hydrate thereof) that is adequate for the intended effect.

Antifungal activity of compounds can be demonstrated by various assays known in the art, for example, by their minimum inhibitory concentration (MIC) against yeasts and minimum effective concentration (MEC) against filamentous molds and dermatophytes in a broth microdilution assay, or in vivo evaluation of the anti-Candida activity in mice, rabbit or guinea pig models. The compounds of Formula (I) provided in the Examples of U.S. Pat. No. 8,188,085 were generally found to inhibit the growth of Candida spp. in the range of <0.03-32 μg/mL. For Candida auris specifically, the distribution of MIC values of ibrexafungerp ranged from 0.0625 μg/mL to 2 μg/mL; the overall mode was 1 μg/mL; and the MIC₅₀ and MIC₉₀ were 0.5 micrograms/ml and 1 micrograms/ml, respectively. (Berkow E L, Angulo D, Lockhart S R, In vitro activity of a novel glucan synthase inhibitor, SCY-078, against clinical isolates of Candida auris, Antimicrob Agents Chemother 61:e00435-17 (2017) https://doi.org/10.128/AAC.00435-17.)

### EXAMPLES

The following examples serve only to illustrate the invention and its practice. The examples are not to be construed as a limitation on the scope or spirit of the invention.

### Example 1

Evaluation of Ibrexafungerp (SCY-078) in the Reduction of Candida auris Skin Burden in a Guinea Pig Model

The purpose of this study was to evaluate of whether orally administered ibrexafungerp was able to reduce Candida auris burden in infected skin.

### Materials and Methods

Guinea pigs (n=5 per group) were randomized to receive 10 or 20 or 30 mg/kg of ibrexafungerp twice daily (BID) by gavage, or vehicle control. Animals received a single dose of prednisolone at 30 mg/kg, subcutaneously, one day prior and one and three days post infection to favor immuno-compromise of the animals and facilitate the development of Candida auris skin infection. A 100 μl cell suspension containing 10⁸ blastospores of Candida auris was applied to an abraded area on the back of the animals. At Day 7, tissue biopsies were examined histologically, and tissue fungal burden was analyzed by colony counts from skin samples. PK bioanalysis of ibrexafungerp plasma concentrations was conducted following the final dose (Day 7).

### Results

Tissue burdens of Candida auris were lower in all treatment groups versus in the vehicle control group. No fungal elements were observed in the biopsy samples from animals treated with ibrexafungerp as opposed to samples from animals in the untreated control group. There were no significant differences in the clinical scores (crusting, inflammation) among the active treatment groups. Animals dosed with 10, 20, or 30 mg/kg BID of ibrexafungerp had plasma exposures (AUC₀₋₂₄) of 2.8, 5.6, and 15 μg*hr/ml.

### CONCLUSIONS

The results from this experimental model showed that treatment with ibrexafungerp reduced the fungal burden in skin infected with Candida auris, when compared to the untreated control, thus supporting a role for ibrexafungerp for decolonization of Candida auris from the skin.

Further, in previous animal models of systemic Candida spp. infection, the exposures needed to achieve efficacy was ˜11.2 μg*hr/ml. (Wring S A et al., Preclinical Pharmacokinetics and Pharmacodynamic Target of SCY-078, a First-in-Class Orally Active Antifungal Glucan Synthesis Inhibitor, in Murine Models of Disseminated Candidiasis, Antimicrob Agents Chemother, 2017 Mar. 24; 61(4). pii: e02068-16. doi: 10.1128/AAC.02068-16. Print 2017 April) In the present study, potent antifungal activity was observed in the skin at doses that resulted in plasma exposures below what was previously reported as being necessary to achieve systemic efficacy, showing that ibrexafungerp has unique attributes and exhibits potent antifungal activity in the skin, and can be used to address the problem of Candida auris colonization of the skin.

### Example 2

Low MIC₅₀ values for ibrexafungerp were found for 102 Candida auris clinical and surveillance isolates from an outbreak in New York. The isolates included C. auris with a variable resistance to antifungal drugs (resistance to one drug in one or two classes of antifungal drugs), multidrug-resistant isolates (resistance to two or more drugs between two classes of antifungal drugs), and pan-resistant isolates (resistance to two or more azoles, all tested echinocandins, and amphotericin B). For the 97 isolates that had variable or multidrug resistance to the other tested antifungal drugs (including fluconazole, voriconazole, itraconazole, isavuconazole, posaconazole, anidulafungin, caspofungin, micafungin, amphotericin B, and flucytosine), the ibrexafungerp MIC₅₀ range was 0.06-0.5 μg/ml; the median and mode for ibrexafungerp MIC₅₀ were each 0.5 μg/ml. There were five pan-resistant C. auris isolates, and all of these were susceptible to ibrexafungerp at a low MIC₅₀ range of 0.12 to 1 μg/ml.

While this invention has been particularly shown and described with references to preferred embodiments thereof, it will be understood in light of the present disclosure by those skilled in the art that various changes in form and details may be made therein without departing from the scope of the invention encompassed by the appended claims.

What is claimed is:

1. A method of decolonizing Candida auris from an anatomic area of a subject colonized by Candida auris, the method comprising administering to the subject a compound of Formula (I): 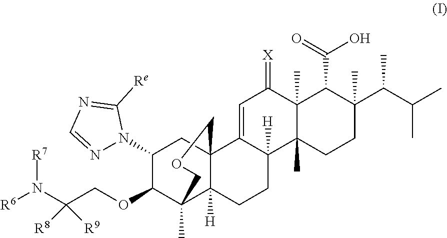
or a pharmaceutically acceptable salt or hydrate thereof, wherein:
X is O or H, H;
R^{e} is C(O)NR^{f}R^{g} or a 6-membered ring heteroaryl group containing 1 or 2 nitrogen atoms wherein the heteroaryl group is optionally mono-substituted on a ring carbon with fluoro or chloro or on a ring nitrogen with oxygen;
R^{f}, R^{g}, R⁶ and R⁷ are each independently hydrogen or C₁-C₃ alkyl;
R⁸ is C₁-C₄ alkyl, C₃-C₄ cycloalkyl or C₄-C₅ cycloalkyl-alkyl;
R⁹ is methyl or ethyl; and
R⁸ and R⁹ are optionally taken together to form a 6-membered saturated ring containing 1 oxygen atom.

2. The method according to claim 1, wherein the anatomic area is skin.

3. The method according to claim 1, wherein the anatomic area is mucosal tissue.

4. The method according to claim 3, wherein the mucosal tissue is respiratory, gastrointestinal, or urinary tract mucosal tissue.

5. The method according to claim 1, wherein the subject is a human subject.

6. The method according to claim 1, wherein the compound of Formula (I) or a pharmaceutically acceptable salt or hydrate thereof is administered orally.

7. The method according to claim 1, wherein the compound of Formula (I) or a pharmaceutically acceptable salt or hydrate thereof is administered intravenously.

8. A method of decolonizing Candida auris from an anatomic area of a subject colonized by Candida auris, the method comprising administering to the subject a compound of Formula (II): 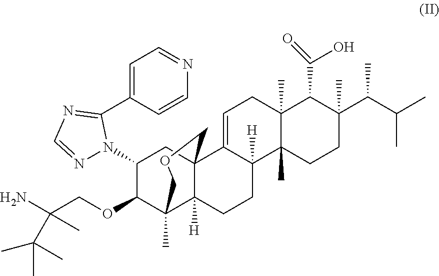
which is (1S,4aR,6aS,7R,8R,10aR,10bR,12aR,14R,15R)-15-[[2-amino-2,3,3-trimethylbutyl]oxy]-8-[(1R)-1,2-dimethylpropyl]-14[5-(4-pyridinyl)-1H-1,2,4-triazol-1-yl]-1,6,6a,7,8,9,10,10a,10b,11,12,12a-dodecahydro-1,6a,8,10a-tetramethyl-4H-1,4a-propano-2H-phenanthro[1,2-c]pyran-7-carboxylic acid,
or a pharmaceutically acceptable salt or hydrate thereof.

9. The method according to claim 8, wherein the anatomic area is skin.

10. The method according to claim 8, wherein the anatomic area is mucosal tissue.

11. The method according to claim 10, wherein the mucosal tissue is respiratory, gastrointestinal, or urinary tract mucosal tissue.

12. The method according to claim 8, wherein the subject is a human subject.

13. The method according to claim 8, wherein the compound of Formula (II) or a pharmaceutically acceptable salt or hydrate thereof is administered orally.

14. The method according to claim 8, wherein the compound of Formula (II) or a pharmaceutically acceptable salt or hydrate thereof is administered intravenously.

15. A method of decolonizing Candida auris from an anatomic area of a subject colonized by Candida auris, the method comprising administering to the subject a compound of Formula (IIa): 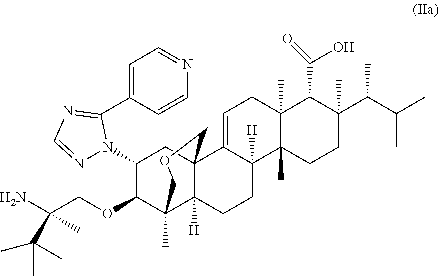
which is (1S,4aR,6aS,7R,8R,10aR,10bR,12aR,14R,15R)-15-[[(2R)-2-amino-2,3,3-trimethylbutyl]oxy]-8-[(1R)-1,2-dimethylpropyl]-14[5-(4-pyridinyl)-1H-1,2,4-triazol-1-yl]-1,6,6a,7,8,9,10,10a,10b,11,12,12a-dodecahydro-1,6a,8,10a-tetramethyl-4H-1,4a-propano-2H-phenanthro[1,2-c]pyran-7-carboxylic acid,
or a pharmaceutically acceptable salt or hydrate thereof.

16. The method according to claim 15, wherein the anatomic area is skin.

17. The method according to claim 15, wherein the anatomic area is mucosal tissue.

18. The method according to claim 17, wherein the mucosal tissue is respiratory, gastrointestinal, or urinary tract mucosal tissue.

19. The method according to claim 15, wherein the subject is a human subject.

20. The method according to claim 15, wherein the compound of Formula (IIa) or a pharmaceutically acceptable salt or hydrate thereof is administered orally.

21. The method according to claim 15, wherein the compound of Formula (IIa) or a pharmaceutically acceptable salt or hydrate thereof is administered intravenously.

22. A method of decolonizing Candida auris from an anatomic area of a human subject colonized by Candida auris, the method comprising administering to the human subject a compound of Formula (IIa): 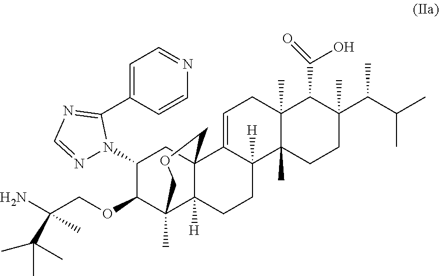
which is (1S,4aR,6aS,7R,8R,10aR,10bR,12aR,14R,15R)-15-[[(2R)-2-amino-2,3,3-trimethylbutyl]oxy]-8-[(1R)-1,2-dimethylpropyl]-14[5-(4-pyridinyl)-1H-1,2,4-triazol-1-yl]-1,6,6a,7,8,9,10,10a,10b,11,12,12a-dodecahydro-1,6a,8,10a-tetramethyl-4H-1,4a-propano-2H-phenanthro[1,2-c]pyran-7-carboxylic acid.

23. The method according to claim 22, wherein the anatomic area is skin.

24. The method according to claim 22, wherein the anatomic area is mucosal tissue.

25. The method according to claim 24, wherein the mucosal tissue is respiratory, gastrointestinal, or urinary tract mucosal tissue.

26. A method of decolonizing Candida auris from an anatomic area of a human subject colonized by Candida auris, the method comprising administering to the human subject a pharmaceutically acceptable salt of a compound of Formula (IIa): 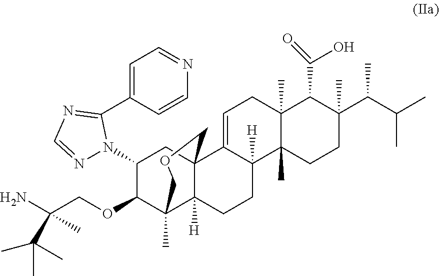
the compound being (1S,4aR,6aS,7R,8R,10aR,10bR,12aR,14R,15R)-15-[[(2R)-2-amino-2,3,3-trimethylbutyl]oxy]-8-[(1R)-1,2-dimethylpropyl]-14[5-(4-pyridinyl)-1H-1,2,4-triazol-1-yl]-1,6,6a,7,8,9,10,10a,10b,11,12,12a-dodecahydro-1,6a,8,10a-tetramethyl-4H-1,4a-propano-2H-phenanthro[1,2-c]pyran-7-carboxylic acid.

27. The method according to claim 26, wherein the anatomic area is skin.

28. The method according to claim 26, wherein the anatomic area is mucosal tissue.

29. The method according to claim 27, wherein the mucosal tissue is respiratory, gastrointestinal, or urinary tract mucosal tissue.

30. A method of decolonizing Candida auris from an anatomic area of a human subject colonized by Candida auris, the method comprising orally administering to the human subject a pharmaceutically acceptable salt of a compound of Formula (IIa): 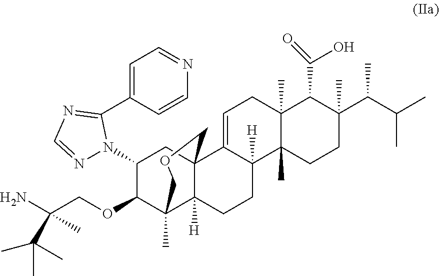
the compound being (1S,4aR,6aS,7R,8R,10aR,10bR,12aR,14R,15R)-15-[[(2R)-2-amino-2,3,3-trimethylbutyl]oxy]-8-[(1R)-1,2-dimethylpropyl]-14[5-(4-pyridinyl)-1H-1,2,4-triazol-1-yl]-1,6,6a,7,8,9,10,10a,10b,11,12,12a-dodecahydro-1,6a,8,10a-tetramethyl-4H-1,4a-propano-2H-phenanthro[1,2-c]pyran-7-carboxylic acid,
wherein the anatomic area is skin or mucosal tissue.

31. The method according to claim 30, wherein the citrate salt of the compound of Formula (IIa) is administered.

32. The method according to claim 30, wherein the pharmaceutically acceptable salt of the compound of Formula (IIa) is administered in a tablet.
